(19) United States
(12) Plant Patent Application Publication
(10) Pub. No.: US 2026/0283057 P1
(43) Pub. Date: Sep. 17, 2026

(54) Sweet Cherry Tree Named 'Prim-bi 3.2'

(50) ;

(71) Applicant: Cerasina GmbH, Kressbronn (DE)

(72) Inventor: Peter STOPPEL, Kressbronn (DE)

(73) Assignee: Cerasina GmbH, Kressbronn (DE)

(21) Appl. No.: 19/563,124

(22) Filed: Mar. 11, 2026

(30) Foreign Application Priority Data
Mar. 12, 2025 (DE) KIA 00131

(51) Int. Cl. A01H 6/74; A01H 5/08

(52) U.S. Cl. CPC A01H 6/7445; A01H 5/08
USPC PLT/181

(57) ABSTRACT

A new and distinct variety of sweet cherry tree, referred to by its cultivar name, ‘Prim-bi 3.2’, is described. The new plant variety exhibits an upright growth habit. The new plant variety produces large fruit which is bi-colored. The parents of the new plant variety are BIGALISER ‘Enjidel’ and ‘Sweet early’.

Latin name of genus and species of plant claimed: Prunus avium (L.) L.

Variety denomination: ‘Prim-bi 3.2’.

### CROSS REFERENCE TO RELATED APPLICATIONS

This application claims priority to Plant Breeders' Right Application No. KIA 00131, which was filed in Germany on Mar. 12, 2025, the contents of which are hereby incorporated by reference for all purposes.

### BACKGROUND OF THE INVENTION

The new plant variety of sweet cherry tree of the present invention was created by controlled breeding during spring flowering in 2009, in a controlled environment in Germany, by artificial pollination wherein two parents were crossed which previously had been studied in the hope that they would contribute the desired characteristics. The female parent (i.e., the seed parent) of the new plant variety was the BIGALISE® ‘Enjidel’ variety (not patented). The male parent (i.e., the pollen parent) of the new plant variety was the ‘Sweet early’ variety (not patented).

The parentage of the new plant variety can be summarized as follows:
BIGALISE® ‘Enjidel’ x ‘Sweet early’

The seeds resulting from the above pollination were harvested in the fall of 2009. The seeds were sown and germinated, seedlings are raised in the spring of 2010, and flowering and fruiting occurred around 2012. These F1 population trees were observed, and selective study resulted in the identification of a single plant of the new plant variety.

The new plant variety has been found to undergo asexual propagation by cuttings. Asexual propagation techniques, such as cuttings, have shown that the characteristics of the new plant variety are homogeneous, stable, and strictly transmissible by such asexual propagation from one generation to another. Accordingly, the new plant variety undergoes asexual propagation in a true-to-type manner.

### SUMMARY OF THE INVENTION

It was found that the new variety of sweet cherry tree of the present invention possesses the following combination of characteristics:
(a) forms an upright growth habit, and
(b) produces large sized fruit.

The new plant variety can be readily distinguished from its ancestors. More specifically, the new plant variety has a stronger upright growth and produces fruit that is bi-colored compared to the ‘BIGALISER ‘Enjidel’ variety (i.e., the seed parent) and the ‘Sweet early’ variety (i.e., the pollen parent). In addition, the new plant variety is S1S2 S-alleles, whereas the BIGALISER ‘Enjidel’ variety is S2S3. Moreover, the new plant variety can be readily distinguished from non-parental related similar varieties. For example, the new plant variety produces fruit that is more kidney-shaped, has a shorter and thicker stem, has a base that is blunter which is almost obcordate, and is slightly less sweet with less sugar content compared to fruit of the ‘Rainier’ variety (not patented).

The new plant variety has been named the ‘Prim-bi 3.2’ variety. The new plant variety has also been referred to as Breeder's Reference: ‘Stop R022’.

### BRIEF DESCRIPTION OF THE PHOTOGRAPHS

The accompanying photographs show, as nearly true as it is reasonably possible to make the same in a color illustration of this character, a typical specimen of the new plant variety and fruit of the new plant variety.

FIG. 1 illustrates specimen of fruit growing on the new plant variety.

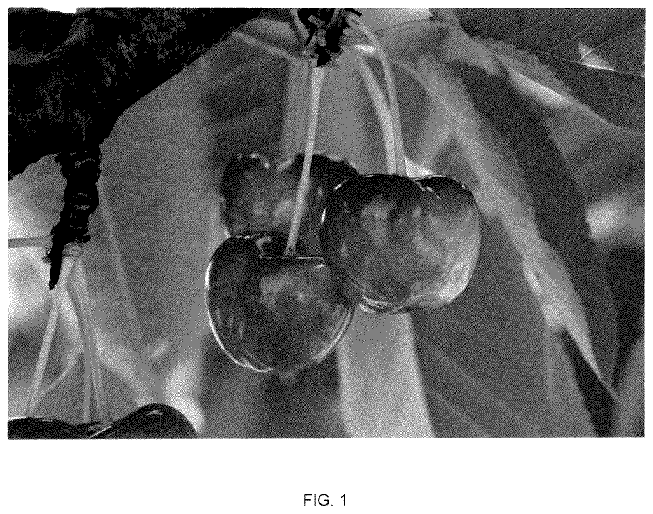

FIG. 2 illustrates specimen of halved fruit of the new plant variety.

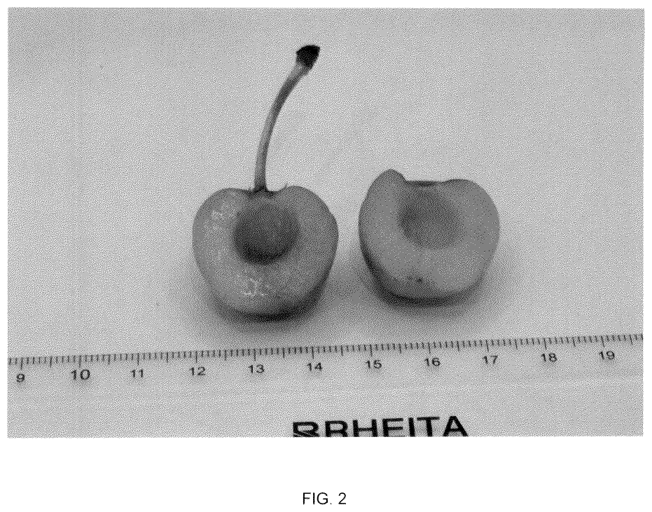

### DETAILED BOTANICAL DESCRIPTION

The chart used in the identification of colors is that of The Royal Horticultural Society (The R.H.S. Colour Chart, 2015 edition). The terminology which precedes reference to the chart has been added to indicate the corresponding color in more common terms. The R.H.S. Colour Chart designation used herein represents the closest color observed on the majority of the specified botanical features. The description is based on seven-years-old specimens of the new plant variety while growing on ‘Gisela 5’ rootstock in Kressbronn, Germany.
Botanical classification: Prunus avium (L.) L. cultivar ‘Prim-bi 3.2’.
Commercial classification: Sweet Cherry Tree.
Tree:
   Vigor.—Strong to very strong.
   Height.—Approximately 4.0 m.
   Width.—Approximately 2.0 m.
   Growth habit.—Strong-medium strong.
   Form.—Semi upright.
   Trunk diameter.—Approximately 10 cm above ground.
   Trunk surface texture.—Smooth with prominent lenticels.
   Lenticel length.—Typically 1.0 cm to 2.0 cm.
   Lenticel width.—Approximately 0.5 cm.
   Lenticel color.—Greyed-Orange Group 167A.
   Lenticel density.—Medium.
   Bark color.—Grey-Brown Group 199A.
Branches:
   Diameter.—Typically 0.5 cm to 1.5 cm.
   Surface.—Average, typical sweet cherry.
   Form.—Typical sweet cherry.
   Bark color.—Greyed-Orange Group 164A.
   Average angle.—Typically 60 degrees to 75 degrees.
   Lenticels length.—Approximately 1.0 mm.
   Lenticels width.—Approximately 1.0 mm.
   Lenticels shape.—Roundish.
   Lenticels color.—Greyed-Yellow Group 161B.
   Lenticels density.—Few, scattered.
Leaves:
   Length.—Typically 13.0 cm to 20.0 cm.
   Width.—Typically 5.0 cm to 8.0 cm.
   Form.—Long, narrow.
   Texture.—Leathery, no crisp feeling.
   Thickness.—Medium.
   Base.—Obtuse.
   Apex.—Pungent.
   Margin.—Serrations regular.
   Color mature leaves upper surface.—Green Group 143A.
   Color mature leaves lower surface.—Green Group 142D.
   Petiole shape.—Narrow.
   Petiole length.—Approximately 3.0 cm.
   Petiole diameter.—Approximately 2.5 mm.
   Petiole color.—Adaxial Red-Purple Group 59A, abaxial Yellow-Green Group 149C.
   Venation type.—Pinnate.
   Veins color upper surface.—Yellow-Green Group 154D.
Flower buds:
   Length.—Approximately 10.0 mm.
   Width.—Approximately 4.0 mm.
   Color.—Reddish.
   Pedicel length.—Typically 7.0 mm to 10.0 mm.
   Pedicel diameter.—Approximately 1.0 mm.
Flowers:
   Bloom timing.—Approximately Apr. 11, 2025.
   Blooming period.—Early to Medium.
   Number of flowers per raceme.—Typically 3 to 7.
   Fragrance.—None.
   Diameter.—Approximately 3.0 cm.
   Petals number.—5.
   Petals length.—Approximately 15.0 mm.
   Petals width.—Approximately 12.0 mm.
   Petals shape.—Rounded ovate.
   Petals margin.—Slightly undulated.
   Petals texture and appearance.—Soft texture.
   Petals color when opening.—Upper and lower surfaces White N999D.
   Petals color fully opened.—Upper and lower surfaces White N999D.
   Petal arrangement.—Overlapping.
   Sepals shape.—Obovate large.
   Sepals margin.—Regular.
   Sepals length.—Approximately 6.0 mm.
   Sepals width.—Approximately 4.0 mm.
   Sepals color.—Upper and lower surfaces are Yellow-Green Group 146C.
   Stamens number per flower.—Approximately 39.
   Filament length.—Typically 3.0 to 11.0 mm.
   Anthers shape.—Round-oval.
   Anthers length.—Approximately 1.0 mm.
   Anthers color.—Greyed-Orange Group N167A.
   Pollen color.—Greyed-Orange Group N167A.
   Pollen amount.—Plentiful.
   Pistils.—Approximately 15.0 mm.
   Style length.—Approximately 12.0 mm.
   Style color.—Yellow-Green Group 145B.
   Stigma shape.—Oval.
   Stigma color.—Yellow-Green Group 152D.
Fruit:
   Shape.—Broad ovate.
   Diameter.—Typically between 25.0 mm to 28.0 mm.
   Fruit stalk length.—Approximately 35.0 mm.
   Fruit stalk thickness.—Approximately 1.0 mm.
   Skin color.—Red Group 41A.
   Skin ground color.—Yellow-Orange Group 21B.
   Skin thickness.—Medium.
   Skin texture.—Smooth.
   Flesh color.—Yellow Group 8C.
   Firmness.—Firm.
   Aroma.—Pleasant cherry flavor.
   Eating quality.—Good.
   Stone length.—Approximately 10.0 mm.
   Stone width.—Approximately 8.0 mm.
   Stone form.—Oval.
   Stone color.—Greyed-Orange Group 173D.
   Stem length.—Approximately 38. mm.
   Stem width.—Approximately 1.0 mm.
   Stem color.—Green Group 138D.
Development:
   Ripening period.—Early to medium.
   Resistance to pathogens.—Cherry leaf roll virus (Nepovirus), Prune dwarf virus (Ilarvirus), Prunus necrotic ringspot virus (Ilarvirus), Tomato black ring virus (Nepovirus (subgroup B)), Tomato ringspot virus (Nepovirus lycopersici), Little cherry virus 1 (Velarivirus nanoavii), Little cherry virus 2 (Ampelovirus nanoavii).

The new ‘Prim-bi 3.2’ variety has not been observed under all possible environmental conditions to date. Accordingly, it is possible that the phenotypic expression may vary somewhat with changes in light intensity and duration, cultural practices, and other environmental conditions.

I claim:

1. A new and distinct variety of sweet cherry tree named ‘Prim-bi 3.2’ substantially as illustrated and described herein.
